(19) United States
(12) Patent Application Publication
(10) Pub. No.: US 2026/0207958 A1
(43) Pub. Date: Jul. 23, 2026

(54) Treatment of Tinnitus, Hearing Loss, Vertigo, Temporomandibular Joint Disorders, and Middle Ear Effusion Using Magnetic Ear Devices

(71) Applicant: Daniel R. Schumaier, Elizabethton, TN (US)

(72) Inventor: Daniel R. Schumaier, Elizabethton, TN (US)

(21) Appl. No.: 19/567,950

(22) Filed: Mar. 16, 2026

Related U.S. Application Data
(63) Continuation-in-part of application No. 18/744,603, filed on Jun. 15, 2024.
(60) Provisional application No. 63/631,465, filed on Apr. 9, 2024.

(51) Int. Cl. A61N 2/00; A61N 2/02

(52) U.S. Cl. CPC A61N 2/004; A61N 2/02

(57) ABSTRACT

Apparatuses and methods are described for improving blood flow and oxygen levels within an ear of a person for treatment of tinnitus, sudden sensorineural hearing loss, vertigo, temporomandibular joint disorder, or middle ear effusion using a head-worn appliance containing magnetic material that generates a magnetic field. The methods include placing the appliance on the person's head so that the magnetic material is in close proximity to a user's ear. The wearable appliance may be a configured as a headband, a headset, or in-ear inserts. If treating both of the person's ears, the wearable appliance should include two sets of magnets disposed on opposite sides of the person's head and in close proximity to the person's inner ears. The wearable appliance is oriented so that the north pole of the magnets faces the person's inner ear, and the south pole of the magnets faces away from the person's inner ear.

### FIELD

This invention relates to the treatment of health conditions using magnetic fields. More particularly, this invention relates to wearable devices that apply magnetic fields near blood vessels in the ears to improve blood flow for treatment of tinnitus, vertigo, sudden sensorineural hearing loss, temporomandibular joint disorder, and middle ear effusion.

### BACKGROUND

Magnetic energy for healing dates back thousands of years. For example, a book published in China around 2000 B.C., entitled “The Yellow Emperor's Book of Internal Medicine,” mentions using magnetic stones to correct health imbalances. Such stones, which contain a naturally occurring mineral called magnetite (Fe304), have been referred to as Lodestones. The ancient Greeks also knew about the healing effects of Lodestones, and Hippocrates, who is considered the Father of Medicine, talked about the healing powers of Lodestones.

Electromagnetic therapy devices are now being used to eliminate pain, improve healing of broken bones, and reverse degenerative diseases. Magnetic therapy is well accepted in such countries as Germany, Russia, China, Japan, and Australia, just to name a few. More and more American studies are confirming the value of magnetic therapy in healing, and magnetic therapy is gaining credibility in the United States.

Magnets are showing results in healing in general, as well as for cuts, broken bones, inflammation, pain, and more. It is well known that negative magnetic fields have a beneficial effect, and positive magnetic fields have a stressful effect. Research has shown that the north (negative) pole of a magnetic field increases tissue oxygenation, relieves pain, reduces swelling, and promotes tissue alkalinization. On the other hand, the south (positive) pole increases swelling, promotes tissue acidity, and decreases tissue oxygenation. Therefore, the polar direction of the magnetic field is critical for healing. Magnetic strength is another critical factor. Both the correct magnetic strength and the correct magnetic pole orientation must be applied to the affected area to be beneficial.

Research by Rongia Tal, a Temple University physicist, has demonstrated that human blood flow can be improved by subjecting it to a magnetic field. Red blood cells contain iron. “A magnetic field polarizes the red blood cells, causing them to link together to form chains, thus streamlining the movement of the blood. Since these chains are larger than the single cells, they flow down the center, reducing friction against the walls of the blood vessels.” Improved blood flow brings more oxygen to a magnetized area.

It is thought that tinnitus is usually caused by an underlying condition, such as age-related hearing loss, an ear injury, or a problem with the circulatory system. For example, tinnitus can be caused by broken or damaged hair cells in the part of the ear that receives sound (cochlea), changes in how blood moves through nearby blood vessels (carotid artery), problems with the joint of the jawbone (temporomandibular joint), and problems with how the brain processes sound. Blood vessel disorders may also cause tinnitus. Conditions that affect blood vessels, such as atherosclerosis, high blood pressure, or kinked or malformed blood vessels, can cause blood to move through veins and arteries with more force. These blood flow changes can cause tinnitus or make tinnitus more noticeable.

For many people, tinnitus can be improved with treatment of the underlying cause. It has also been treated by masking the noise, thereby making tinnitus less noticeable. Research has been conducted in the use of repetitive transcranial magnetic stimulation (rTMS) to treat tinnitus. This involves delivering short magnetic pulses to the brain using an electromagnetic coil. Because preliminary trials of rTMS have yielded mixed results, researchers are studying the ideal coil placement and frequency of treatments.

Because all hearing is centered on the cochlea, the blood supply provided to the cochlea via the labyrinthine artery (internal auditory artery) is critical. Interruption of this blood flow can cause damage to the stereocilia, resulting in partial to sudden sensorineural hearing loss, tinnitus, and vertigo. Loud noises for prolonged periods of time can also cause damage to the stereocilia due to a decrease in blood flow and oxygen levels, and the inability of the cells to get rid of waste products. Also, an interruption of the blood flow to the vestibular portion of the inner ear can cause vertigo and dizziness.

Each year, more than 66,000 people in the United States experience a sudden sensorineural hearing loss (SSHL). SSHL is experienced as a sudden loss of most or all of the hearing in one ear and is thought to be caused by a sudden blockage of the blood supply to the cochlea provided by the labyrinthine artery. SSHL is generally defined as hearing loss of at least 30 decibels occurring over at least three consecutive audiometric frequencies and lasting at least three days. Those experiencing SSHL may also experience tinnitus, dizziness or vertigo. Any pathology that results in vascular occlusion of the labyrinthine artery can result in SSHL. SSHL has also been associated with decreased oxygen tension in the perilymph, which is an extracellular fluid located within the scala tympani and scala vestibuli of the cochlea. There is also evidence that iron-deficiency anemia may increase the risk for SSHL. Studies have shown that hyperbaric oxygen therapy can increase oxygen tension in the perilymph and restore hearing in a significant number of patients with SSHL. Studies have also shown that the three most promising treatments for SSHL include corticosteroids, hyperbaric oxygen therapy, and vasodilators.

Thus, the vascular system in the inner ear is very critical. In addition to traditional medical treatments, such as those involving corticosteroids, hyperbaric oxygen therapy, and vasodilators, a treatment option is needed to help to increase the blood supply to and blood oxygen levels in the inner ear to maximize the chance of recovery. The needed treatment option should be implemented using comfortable wearable devices that apply magnetic fields near blood vessels in the ears to improve blood flow and increase blood oxygen levels.

### SUMMARY

There are three major problems that may occur with poor blood flow and/or oxygen levels in the inner ear: sudden sensorineural hearing loss, tinnitus, and vertigo (dizziness). Evidence suggests that these issues may be improved through magnetic therapy directed at the inner ear.

Electromagnetism (aka magnetism) does not relate only to iron and other metals. Almost anything can be magnetized. Magnetism occurs on an atomic level in which electrons orbit around the nucleus of the atom. These same electrons spin like the Earth, creating miniature magnetic fields with north and south poles. If all electrons spin in the same direction, magnetism results from an alignment of electrons. Magnetic field power increases as more electrons are aligned.

All magnets have two poles: a negative (north) pole and a positive (south) pole. Most permanent magnets contain one or two magnetizable metals, such as iron oxide and the rare earth metal neodymium. The strength of a magnet is measured in units of Gauss. Earth has a magnetic field that produces electromotive energy. One Gauss is equivalent to about twice the average of Earth's magnetic field. Earth's magnetic field has been dwindling over the last 500 years, which has caused a loss of Gauss strength, a deficiency which can cause medical problems such as stiffness, headaches, dizziness, and the loss of normal healing.

A negative magnetic field favorably affects cell function, hormone production, enzyme activity, to name a few. Negative magnetic fields normalize blood pH levels, reduce cellular swelling, and release molecular oxygen. It is well known that the application of a magnetic negative pole to areas of the body increases blood flow and flow of the lymph system.

With regard to magnetic treatments, evidence suggests that continuous application of low-intensity magnetic fields to blood vessels in the inner ears can improve blood flow and oxygen levels, thereby reducing tinnitus. The inner ear also has a vestibular system that uses the same fluids which may respond with improved blood flow and oxygen levels. The blood supply to the inner ears is also critical to the health of the hair cells for normal hearing. Again, magnetic therapy may improve the blood supply to the inner ear, thereby reducing the loss of these hair cells that cause sudden sensorineural hearing loss and tinnitus.

Embodiments of the invention described herein provide an apparatus for directing a magnetic field to a user's inner ear to improve blood flow and oxygen levels for treatment of one or all of tinnitus, sudden sensorineural hearing loss, vertigo, temporomandibular joint disorder, and middle ear effusion. One embodiment of the apparatus comprises a headband configured to wrap around the user's head and one or more housing assemblies attached to the headband. Each housing assembly includes a base portion and a cover portion. The base portion has a pair of opposing lug portions for slidingly receiving the headband. The cover portion is configured to attach to the base portion, thereby forming a cavity between the base portion and the cover portion. Secured within the cavity is magnetic material configured such that the magnetic field it generates is directed to the inner ear of the user when the housing assembly is disposed adjacent the user's ear.

The magnetic material is oriented within the housing assembly such that the north pole of the magnetic material is directed toward the user's ear, and the south pole is directed away from the user's ear.

In some embodiments, the base and cover portions of the housing assembly are oval-shaped, and the lug portions are disposed on opposing sides of the base portion in alignment with the minor axes of the oval.

In some embodiments, the base portion includes first cradle protrusions that are disposed in a circular arrangement on a central portion of the inner surface of the base portion. The magnetic material comprises one or more circular disks that are disposed between and are at least partially encircled by the first cradle protrusions. The cover portion includes second cradle protrusions extending from a central portion of the inner surface of the cover portion. The second cradle protrusions, which are also disposed in a circular arrangement, are configured to have a snap-fit engagement with the first cradle protrusions.

In some embodiments, the one or more circular disks comprise neodymium.

In some embodiments, the one or more circular magnetic disks generate a magnetic flux density ranging from about 580 Gs to about 1390 Gs.

In some embodiments, the magnetic material comprises an electromagnet.

In some embodiments, the headband is configured to position each housing assembly over the corresponding pinna of the user's ear.

Also described herein are other embodiments that attach in or behind a user's ear to direct a magnetic field to the inner ear to improve blood flow and oxygen levels for treatment of one or all of tinnitus, sudden sensorineural hearing loss, vertigo, temporomandibular joint disorder, and middle ear effusion. Some of these embodiments comprise an outer shell, magnetic material disposed within the outer shell, and a sound tube passing through the magnetic material. The outer shell has an outer profile that is custom-molded to fit within portions of the user's ear. The sound tube has an outer opening that receives sound from outside the user's ear and an inner opening through which the sound passes into the user's ear canal.

In some embodiments, the outer shell comprises an in-canal portion and an in-concha portion. The in-canal portion is configured to be disposed within the user's ear canal, and the in-concha portion is configured to be disposed within the user's ear concha. These embodiments include a faceplate that is attached to the in-concha portion of the outer shell to contain the magnetic material disposed therein.

In some embodiments, the outer opening of the sound tube is disposed in the faceplate, and the inner opening of the sound tube is disposed in the in-canal portion of the outer shell.

In some embodiments, the magnetic material comprises neodymium magnetic spheres.

In some embodiments, the neodymium magnetic spheres are encased in a filler material within the outer shell.

In some embodiments, the filler material comprises a hardened acrylic resin.

In some embodiments, wherein the filler material comprises silicone.

Also described herein are embodiments that attach around a user's ear to direct a magnetic field to the inner ear of a user to improve blood flow and oxygen levels for treatment of one or all of tinnitus, sudden sensorineural hearing loss, vertigo, temporomandibular joint disorder, and middle ear effusion. Some of these embodiments comprise an outer shell having an outer profile configured to fit behind the pinna of the user's ear, and magnetic material disposed within the outer shell. In some of these embodiments, the outer shell comprises a loop configured to at least partially encircle the user's outer ear.

In another aspect, methods are described herein for improving blood flow and oxygen levels within an ear of a user for treatment of tinnitus, sudden sensorineural hearing loss, vertigo, temporomandibular joint disorder, or middle ear effusion using a head-worn appliance containing magnetic material that generates a magnetic field. In some embodiments, the methods include:
placing the head-worn appliance on the user's head such that the magnetic material is in close proximity to one or both ears of the user;
orienting the head-worn appliance such that the north pole of the magnetic material is directed toward the user's ear, and the south pole of the magnetic material is directed away from the user's ear; and
the user wearing the head-worn appliance for an extended period of time.

In some embodiments, the extended period of time comprises at least two hours per day over a period of at least two weeks. The appliance can be worn longer if the user prefers.

In some embodiments, the magnetic material generates a magnetic flux density ranging from about 580 Gs to about 1390 Gs.

Also described herein are methods for improving blood flow and oxygen levels within an ear of a user for treatment of one or more of tinnitus, sudden sensorineural hearing loss, vertigo, temporomandibular joint disorder, and middle ear effusion using an in-ear appliance containing magnetic material that generates a magnetic field. In some embodiments the methods comprise:
inserting the in-ear appliance into the user's ear such that the magnetic material is in close proximity to the inner ear of the user;
orienting the in-ear appliance such that the north pole of the magnetic material is directed toward the user's inner ear, and the south pole of the magnetic material is directed away from the user's inner ear; and
the user wearing the wearable appliance for an extended period of time.

In some embodiments, the inserting step comprises inserting the in-ear appliance into the ear canal of the user.

In some embodiments, the inserting step comprises placing the in-ear appliance into the ear concha of the user.

In yet another aspect, a preferred embodiment described herein is directed to an apparatus for directing a magnetic field to an inner ear of a user to improve blood flow and oxygen levels for treatment of tinnitus, sudden sensorineural hearing loss, vertigo, temporomandibular joint disorder, and middle ear effusion. The apparatus includes a housing assembly configured to be positioned over a pinna of the user's ear, and an ear mold attached to the housing assembly. The housing assembly includes a base portion, a cover portion attached to the base portion, and a first cavity that is formed between the base portion and the cover portion. Disposed within the first cavity is first magnetic material having a north pole that is continuously directed toward a concha of the user's ear and a south pole that is continuously directed away from the concha of the user's ear when the housing assembly is disposed over the pinna of the ear. The ear mold includes an outer portion that is disposed adjacent the cover portion of the housing assembly and configured to be disposed within the user's ear concha. A second cavity is disposed within the outer portion of the ear mold, and second magnetic material is disposed within the second cavity. The second magnetic material has a north pole that is continuously directed toward the user's inner ear and a south pole that is continuously directed away from the user's inner ear when the outer portion is disposed within the concha of the user's ear. The ear mold also includes an inner portion that is attached to the outer portion. The inner portion is sized and configured to be at least partially received within the user's ear canal. A force of magnetic attraction between the first magnetic material in the housing assembly and the second magnetic material in the ear mold maintains the attachment of the housing assembly to the ear mold.

In some embodiments, the inner portion of the ear mold comprises a connector post, and an ear tip is attached to the connector post. The ear tip includes a compliant outer structure disposed around a tubular core. The compliant outer structure is configured to be received within the ear canal of the user.

In some embodiments, the compliant outer structure of the ear tip comprises viscoelastic foam.

In some embodiments, the connector post of the ear mold has external threads, and the tubular core of the ear tip has internal threads corresponding to the external threads of the connector post.

In some embodiments, the outer portion of the ear mold is shaped and dimensioned to provide a universal fit that is applicable to more than one user.

In some embodiments, the base portion includes first cradle protrusions extending from a central portion of an inner surface of the base portion in a circular arrangement. The first magnetic material may comprise one or more circular magnetic disks that are disposed between and at least partially encircled by the first cradle protrusions. The cover portion includes second cradle protrusions extending from a central portion of an inner surface of the cover portion. The second cradle protrusions are disposed in a circular arrangement and are configured to have a snap-fit engagement with the first cradle protrusions.

In some embodiments, the circular magnetic disks comprise neodymium, and generate a magnetic flux density ranging from about 580 Gs to about 1390 Gs.

In some embodiments, the first magnetic material comprises an electromagnet.

In some embodiments, the second magnetic material comprises a stack of multiple annular ring magnets.

In some embodiments, the stack of multiple annular ring magnets generate a magnetic flux density ranging from about 200 Gs to about 880 Gs.

In some embodiments, the force of magnetic attraction between first magnetic material and the second magnetic material removably secures the ear mold to the housing assembly. The force of magnetic attraction is preferably strong enough to maintain secure attachment of the housing assembly to the ear mold as the outer portion of the ear mold is placed into the user's ear concha and the inner portion of the ear mold is inserted into the user's ear canal, but not strong enough to prevent the user from intentionally pulling the ear mold and the housing assembly apart when desired.

A method for using the apparatus to improve blood flow and oxygen levels within the ear of the user for treatment of tinnitus, sudden sensorineural hearing loss, vertigo, temporomandibular joint disorder, or middle ear effusion includes the following steps:
inserting the inner portion of the ear mold into the user's ear canal and inserting the outer portion of the ear mold into the user's ear concha such that the second magnetic material is in close proximity to the user's inner ear;
attaching the housing assembly to the ear mold such that the housing assembly is adjacent the pinna of the user's ear and the first magnetic material is in close proximity to the user's ear concha;
orienting the appliance such that the north poles of the first and second magnetic materials are directed toward the user's inner ear, and the south poles of the first and second magnetic materials are directed away from the user's inner ear; and
the user wearing the appliance for an extended period of time.

In some embodiments, the step of attaching the housing assembly to the ear mold is performed either before or after the steps of inserting the inner portion of the ear mold into the user's ear canal and inserting the outer portion of the ear mold into the user's ear concha.

### BRIEF DESCRIPTION OF THE DRAWINGS

Other embodiments of the invention will become apparent by reference to the detailed description in conjunction with the figures, wherein elements are not to scale so as to more clearly show the details, wherein like reference numbers indicate like elements throughout the several views, and wherein:

FIG. 1 depicts an over-the-ear (OTE) magnetic ear therapy apparatus according to a first embodiment;

FIGS. 2A and 2B depict front and rear views, respectively, of a magnet housing assembly of the OTE magnetic ear therapy apparatus according to the first embodiment;

FIGS. 3A and 3B depict front and rear exploded views, respectively, of the magnet housing assembly according to the first embodiment;

FIGS. 4A and 4B depict a front view and a cross-section view, respectively, of the magnet housing assembly according to the first embodiment;

FIG. 5 depicts the inner surface of a base portion of the magnet housing assembly according to the first embodiment;

FIG. 6 depicts the inner surface of a cover portion of the magnet housing assembly according to the first embodiment;

FIG. 7 depicts lines of magnetic flux generated by the magnet housing assembly according to the first embodiment;

FIGS. 8A and 8B depict examples of electromagnetic circuits for generating magnetic fields according to the first embodiment;

FIG. 9 depicts an in-the-ear (ITE) ear mold of a magnetic ear therapy apparatus according to a second embodiment;

FIG. 10 depicts an ITE ear mold of a magnetic ear therapy apparatus according to the second embodiment disposed in a user's ear;

FIG. 11 depicts an ITE ear mold of a magnetic ear therapy apparatus without a sound tube according to a third embodiment;

FIG. 12 depicts ITE ear molds of magnetic ear therapy apparatuses for left and right ears without a sound tube according to the third embodiment;

FIG. 13A depicts a completely-in-the-canal (CIC) ear mold of a magnetic ear therapy apparatus according to a fourth embodiment;

FIG. 13B depicts an in-the-canal (ITC) ear mold of a magnetic ear therapy apparatus according to a fifth embodiment;

FIG. 13C depicts an ITE ear mold of a magnetic ear therapy apparatus according to the second embodiment;

FIG. 13D depicts a behind-the-ear (BTE) housing assembly of a magnetic ear therapy apparatus according to a sixth embodiment;

FIGS. 14A and 14B depict an around-the-ear housing assembly of a magnetic ear therapy apparatus according to a seventh embodiment; and

FIGS. 15-20 depict a combination over-the-ear (OTE) and in-the-canal (ITC) magnetic ear therapy apparatus according to an eighth embodiment.

### DETAILED DESCRIPTION

Embodiments described herein are directed to wearable structures, also referred to herein as magnetic ear therapy apparatuses, that are worn over the ear, in the ear canal, in the concha, behind the ear, around the ear, or in combinations thereof.

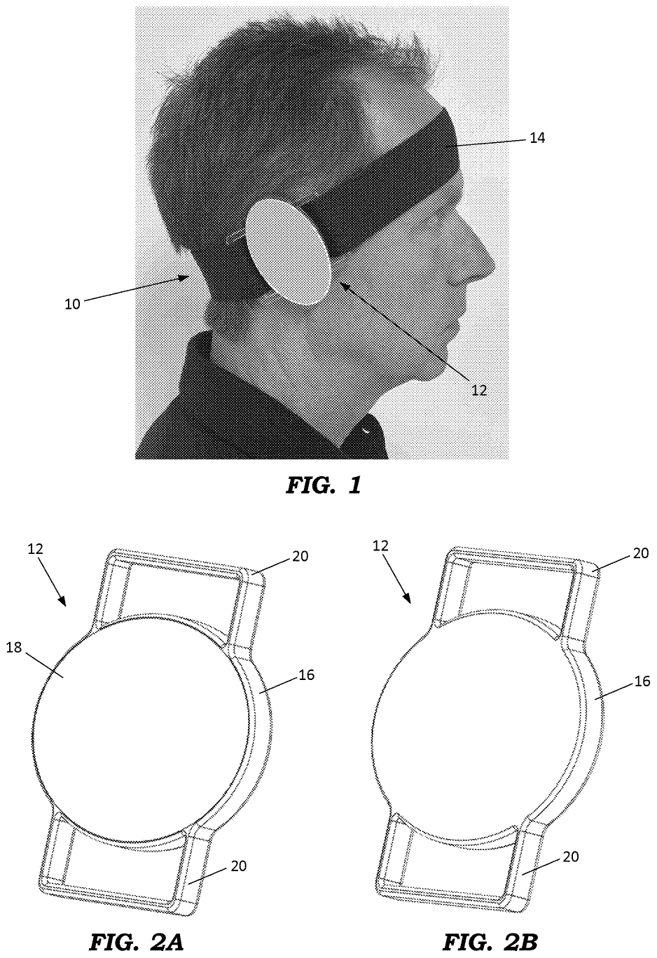

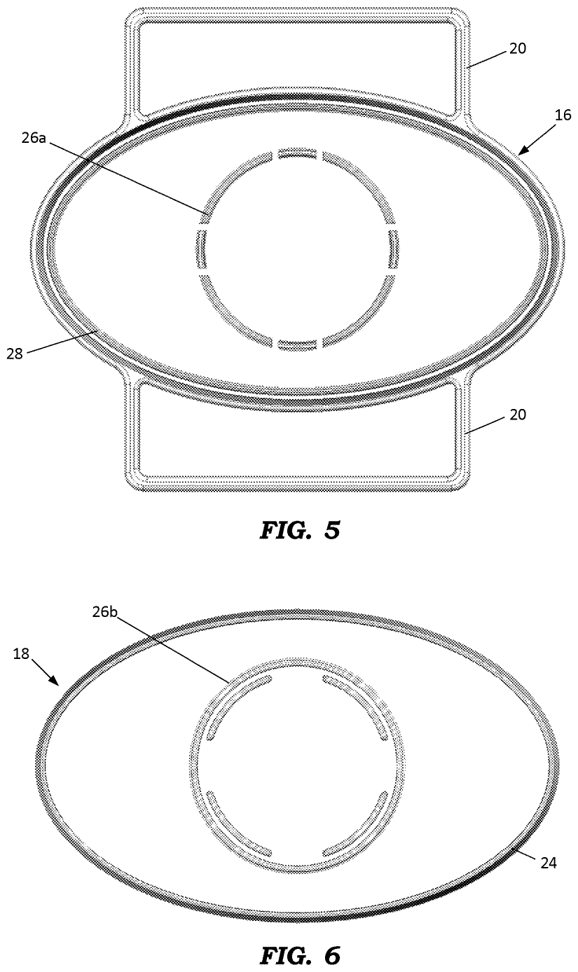

A first embodiment of a magnetic ear therapy apparatus 10 is depicted in FIGS. 1-6. As shown in FIG. 1, a pair of magnet housing assemblies 12 are attached to an elastic headband 14 worn around a user's head. The headband 14 and each housing assembly 12 are positioned such that the housing assemblies 12 are disposed over or adjacent the user's ears. Each magnet housing assembly 12 includes a base portion 16, a cover portion 18, lug portions 20 through which the headband 14 passes, and magnetic material 22 disposed between the base portion 16 and the cover portion 18. It will be appreciated that FIG. 1 shows one of the two housing assemblies 12, the other being disposed on the other side of the user's head.

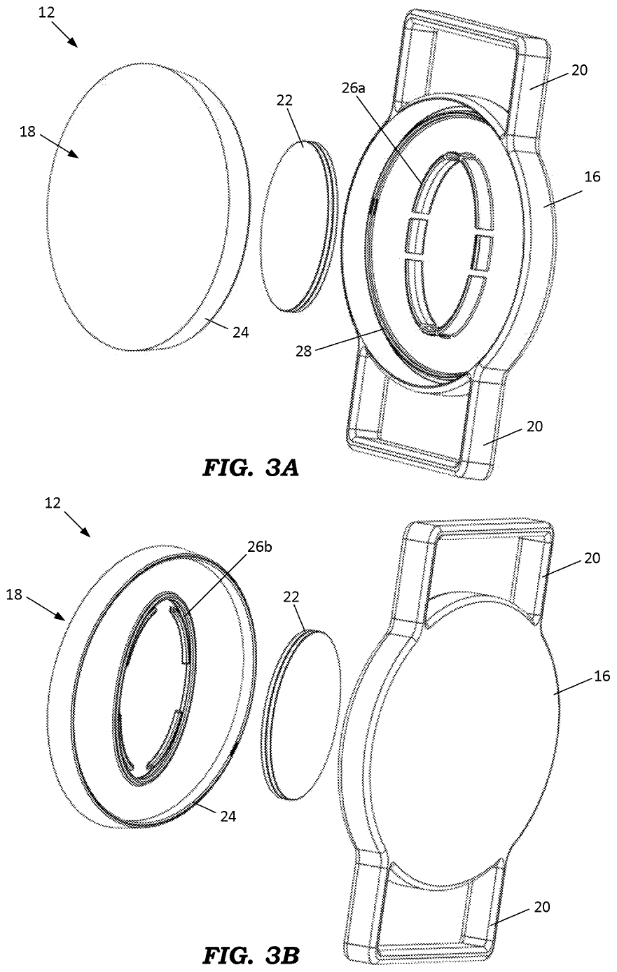

In a preferred version of the first embodiment, the outer perimeter of the base portion 16 is oval-shaped and sized to substantially cover the pinna of the user's ear, with the lug portions 20 extending from the opposing wider sides of the base portion 16. It will be appreciated that the outer perimeter of the base portion 16 could have other shapes in other embodiments, such as rectangular or circular. As shown in FIGS. 3A, 4B and 5, central magnet cradle protrusions 26a and a perimeter protrusion 28 extend inwardly from the inside surface of the base portion 16. Preferably, the base portion 16, the lug portions 20, the central magnet cradle protrusions 26a, and the perimeter protrusion 28 are integrally formed as a single piece of molded plastic.

In the preferred embodiment, the cover portion 18 has an outer perimeter that is also oval-shaped and sized to match the outer perimeter of the base portion 16. As depicted in FIGS. 3A, 3B, 4B and 6, the cover portion 18 has an outwardly extending lip 24 configured to be received within a matching channel formed by the perimeter protrusion 28 along the inside edge of the base portion 16. The cover portion 18 also includes central magnet cradle protrusions 26b extending inwardly from its inside surface. Preferably, the cover portion 18, the lip 24 and the central magnet cradle protrusions 26b are integrally formed as a single piece of molded plastic.

In a preferred embodiment, the magnetic material 22 comprises one or more disc-shaped nickel-plated neodymium magnets, each having a diameter of 1.0 inch and thickness of 0.06 inch. One such magnet alone has a magnetic flux density of about 580 Gs. In the embodiment depicted in FIGS. 3A-3B, the magnetic material 22 includes two magnets having a combined magnetic flux density of about 1090 Gs. Three of the magnets combined have a magnetic flux density of about 1390 Gs. In various embodiments, one, two or more magnets may be provided depending on the type of treatment needed. It will be appreciated that other shapes and arrangements of magnets may be provided, depending on the type of treatment needed. In one embodiment, multiple magnets may be provided, and the user has the option to use one, two or more magnets to achieve the magnetic flux density that is most effective for treatment of the user's tinnitus, vertigo or SSHL. In these embodiments, the cover portion 18 is removable from the base portion 16 so that the user may have access to insert the desired number of magnets.

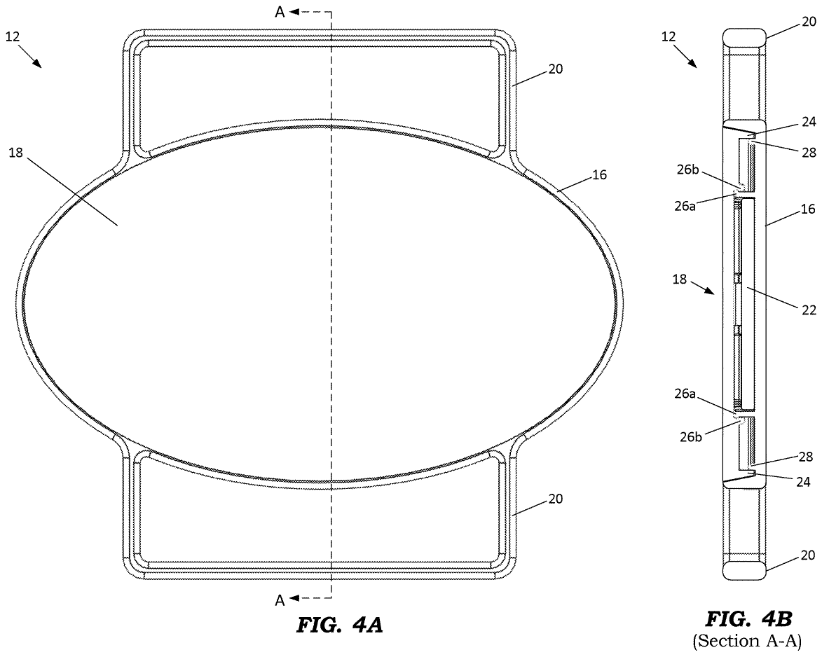

As depicted in FIG. 4B, the central magnet cradle protrusions 26a in the base portion 16 engage the central magnet cradle protrusions 26b in the cover portion 18 in a snap-fit arrangement such that the magnetic material 22 is sandwiched between the base portion 16 and the cover portion 18, and within the central magnet cradle protrusions 26a and 26b.

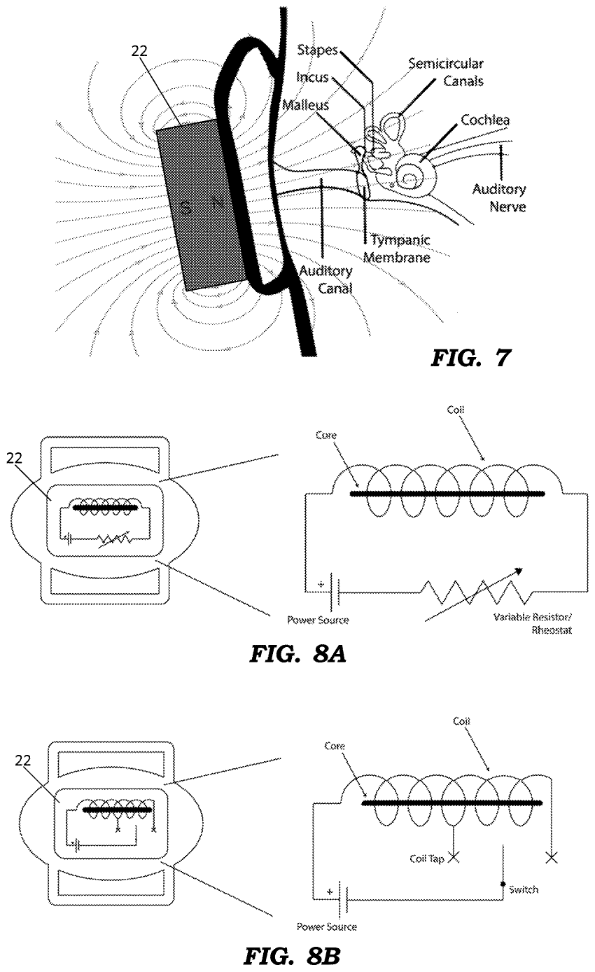

FIG. 7 depicts an example of lines of magnetic field flux generated by the preferred embodiment of the magnetic ear therapy apparatus 10. As illustrated, the magnetic fields emanating from the north pole of the magnetic material 22 are directed to the structures of the user's inner ear to provide the benefits discussed herein, including improved blood flow and increased oxygen levels.

In some embodiments, the magnetic material 22 comprises an electromagnet for generating the magnetic field. In such embodiments, examples of which are depicted in FIGS. 8A and 8B, the user may adjust the magnetic flux density by controlling the voltage level provided to an electromagnetic coil. As illustrated, a switch or other control circuit may be provided to control a rheostat or to select a tap of the coil to control the voltage. Those skilled in the art will appreciate that digital control circuits could also be implemented for this purpose, and the control function may be provided wirelessly, such as via Bluetooth using an app on a mobile device such as a smart phone.

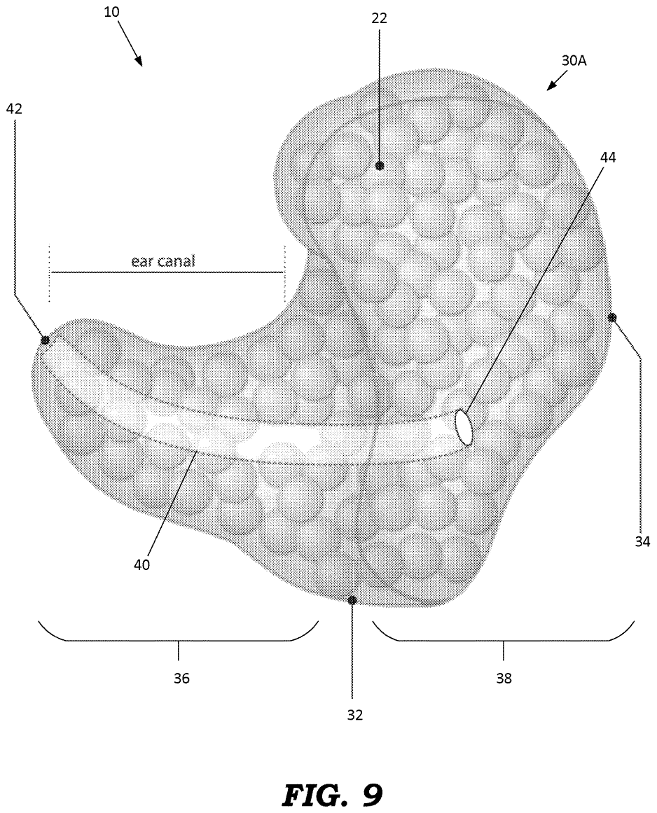

In alternative embodiments, the magnetic ear therapy apparatus 10 comprises an ear mold 30A that encases magnetic materials 22, such as magnetic spheres, and that place the magnetic materials 22 in close proximity to blood vessels in the user's ear when worn as described herein. For example, a second embodiment of the magnetic ear therapy apparatus 10, as depicted in FIGS. 9, 10 and 13C, comprises an ITE ear mold 30A that includes a hollow outer shell 32 in which a magnetic material 22 is enclosed that is capped by a faceplate 34. The outer shell 32 includes an in-canal portion 36 that is disposed within the user's ear canal when worn, and an in-concha portion 38 disposed in the user's ear concha. In a preferred embodiment, the outer shell 32 is formed based on an impression made of the individual user's ear canal and concha, as may typically be done in the manufacturing of custom hearing aid housings. The impression is used to craft the custom-fit outer shell 32.

The magnetic material 22 may comprise magnetic spheres, such as neodymium spheres having a diameter of 3 mm. The spheres are preferably encased in a filler within the outer shell. The filler may be a hardened acrylic resin or a softer material such as silicone. In an alternative embodiment, the magnetic material 22 is a flowable ferromagnetic filling that can be magnetized after the outer shell 32 is filled.

Disposed within the outer shell 32 is a sound tube 40 having an inner opening 42 at the end of the in-canal portion 36 of the outer shell 32 and an outer opening 44 in the faceplate 34 that covers the in-concha portion 38 of the outer shell 32. The sound tube 40 allows sound to travel from outside the faceplate 34 through to the ear canal, so that the user's hearing is unimpeded. An alternative embodiment may include slits or channels on the outside surfaces of the outer shell 32 to allow sound to travel around the outer shell into the ear canal.

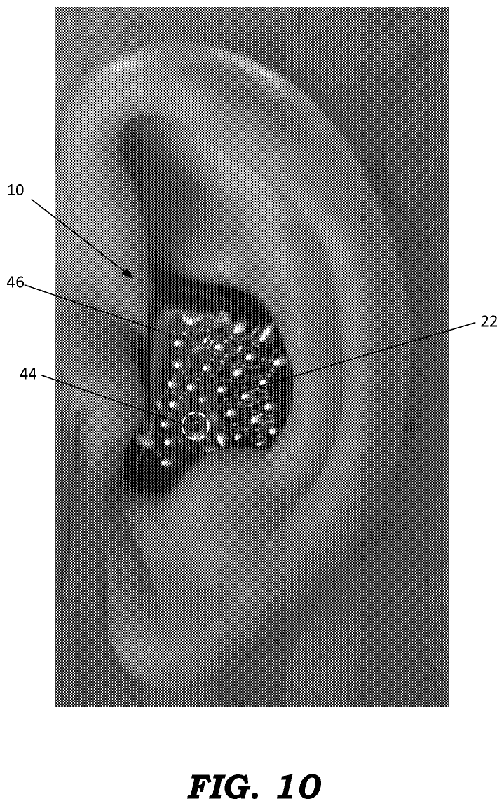

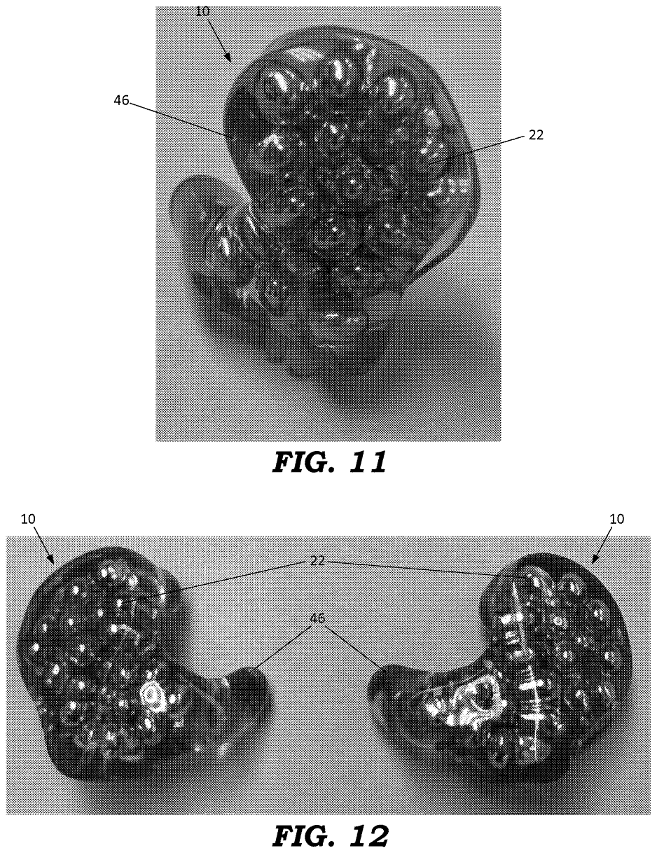

FIGS. 10, 11 and 12 depict ITE ear mold embodiments of the magnetic ear therapy apparatus 10 in which the magnetic material 22 (such as magnetic spheres) are embedded in acrylic resin 46. It will be noted that no sound tube is shown in the embodiments of FIGS. 11 and 12.

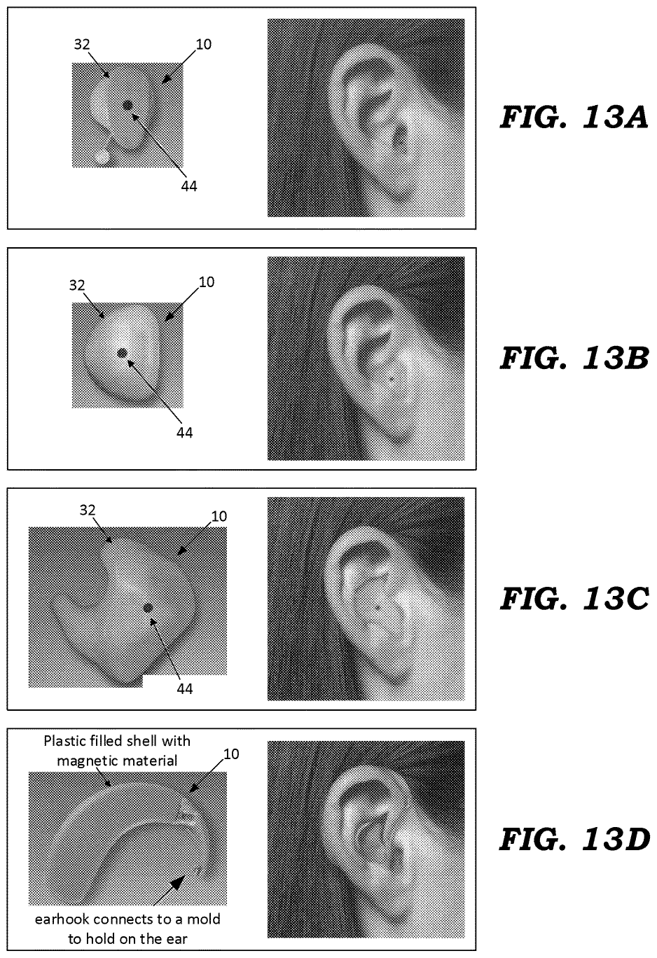

FIGS. 13A, 13B, 13C and 13D depict, respectively, a CIC embodiment of the magnetic ear therapy apparatus 10 having a pull-string for removal, an ITC embodiment, the ITE embodiment, and a BTE embodiment. Preferably, the BTE embodiment is attached by a custom earpiece that is molded to fit the shape of the user's outer ear.

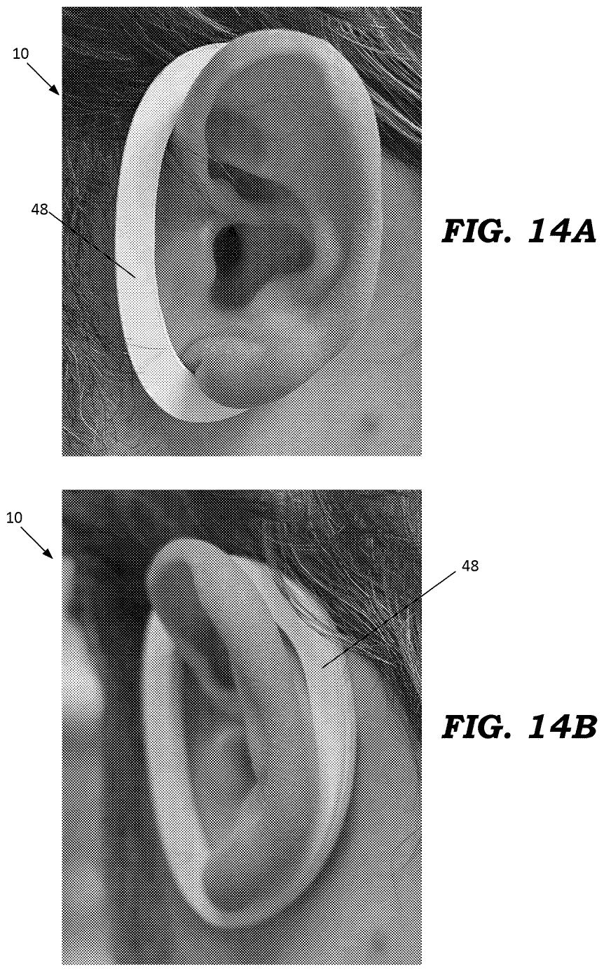

FIGS. 14A and 14B depict an around-the-ear housing assembly 48 in which magnetic material is encased that completely surrounds a user's ear. This version could be worn alone or in combination with the versions depicted in FIGS. 13A, 13B and 13C.

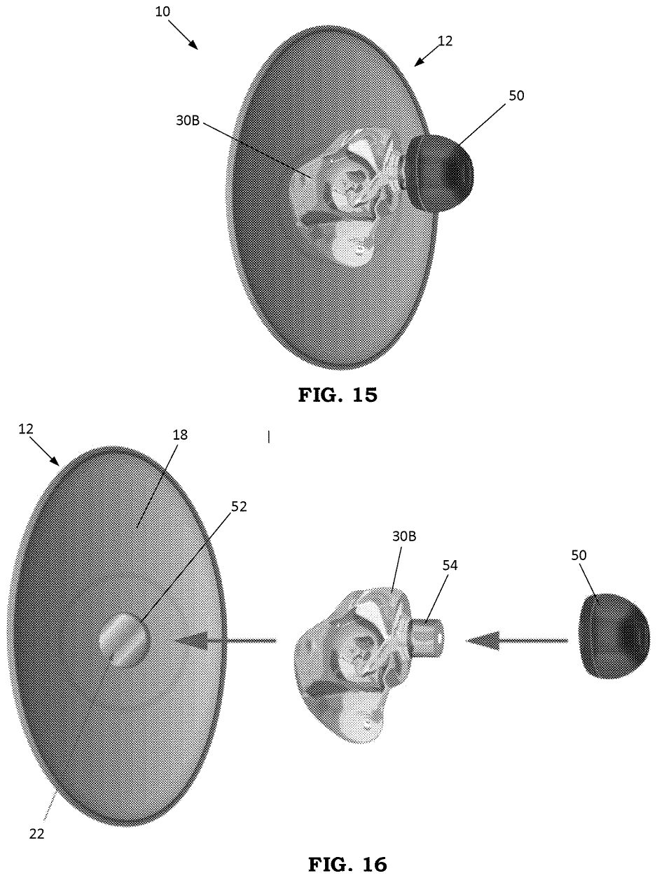

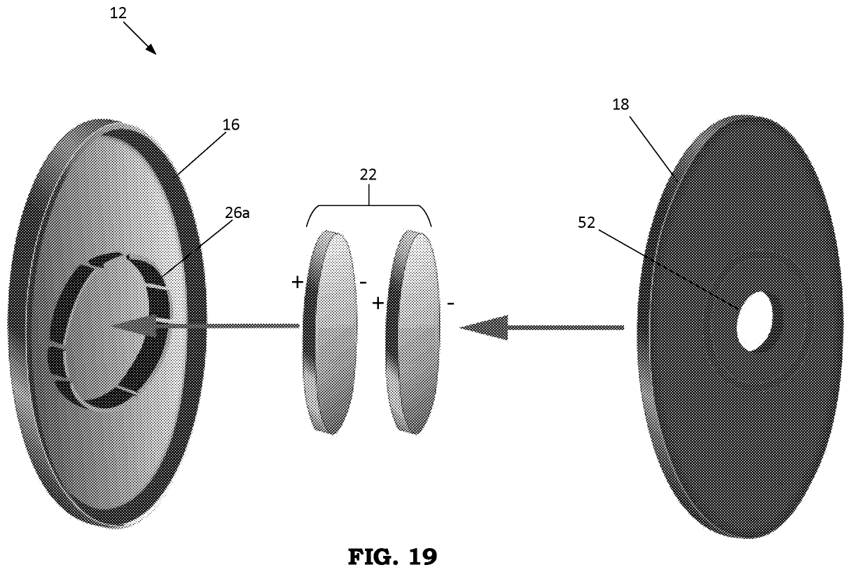

As shown in FIGS. 15-20, an alternative embodiment of the magnetic ear therapy apparatus 10 combines a universal-fit version of an in-the-ear (ITE) ear mold 30B with an over-the-ear (OTE) magnet housing assembly 12. In this embodiment, the magnet housing assembly 12 is structurally similar to the housing assembly depicted in FIGS. 1, 2A-2B, 3A-3B, 4A-4B, 5, and 6, except for the lack of the lug portions 20 and the addition of an aperture 52 in the center of the cover portion 18. The aperture 52 exposes the magnetic material 22 disposed between the base portion 16 and the cover portion 18.

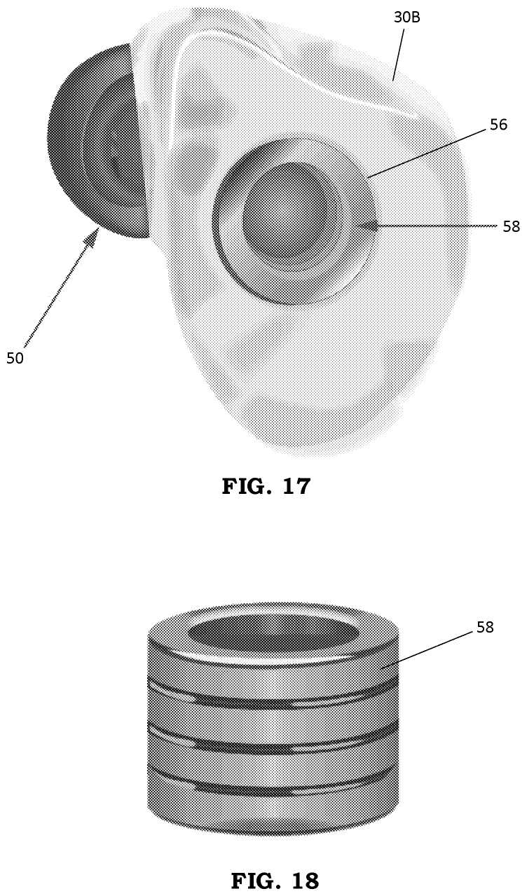

The earmold 30B of this alternative embodiment is also structurally similar to the ear mold 30A depicted in FIGS. 9-12 and 13B, except that, instead of magnetic spheres, the magnetic material in the ear mold 30B includes a stack of four annular ring magnets 58 that are disposed within a cylindrical cavity 56, as shown in FIGS. 17 and 18. It will be appreciated that more or fewer ring magnets 58 may be provided in other embodiments. The housing assembly 12 is removably secured to the ear mold 30B based on the magnetic attraction between the ring magnets 58 in the ear mold 30B and the magnetic material 22 in the housing assembly 12. Preferably, the force of magnetic attraction between the magnets 58 in the ear mold 30B and the magnets 22 in the housing assembly 12 is great enough to keep the housing assembly 12 securely attached to the ear mold 30B as it is placed into a user's ear concha and the ear tip 50 into the user's ear canal, but not so strong as to prevent the user from intentionally pulling the ear mold 30B and the housing assembly 12 apart when desired.

Other differences between the ear mold 30B depicted in FIGS. 15-17 and 20 and the ear mold 30A depicted in FIGS. 9-12 and 13B are in the structure of the in-canal portion 36. As shown in FIGS. 15-17, the ear mold 30B includes a removable universal-fit foam ear tip 50 instead of a custom-molded acrylic tip. In a preferred embodiment, the ear tip 50 comprises a Comply™ foam canal ear tip having a soft memory foam outer structure that surrounds a cylindrical core. The core of the ear tip 50 is preferably internally threaded for screwing onto an externally threaded cylindrical connector post 54 on the ear mold 30B. In other embodiments, an interference fit secures the ear tip 50 to the connector post 54.

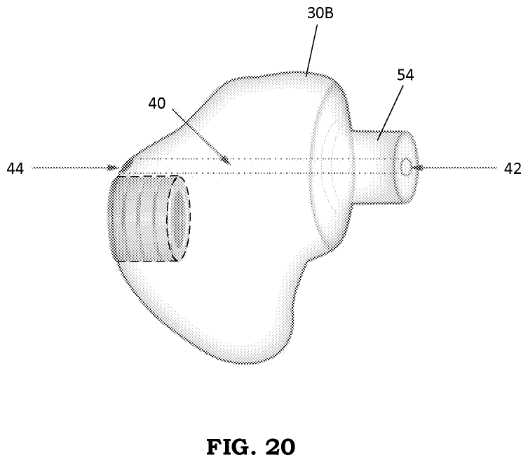

FIG. 20 depicts an embodiment having a sound tube 40 is disposed within the ear mold 30B. The sound tube 40 has an inner opening 42 at the end of the connector post 54 and an outer opening 44 at the opposite end of the ear mold 30B. The sound tube 40 allows sound to travel from outside the ear mold 30B through to the ear canal, so that the user's hearing is unimpeded.

One advantage of the embodiment depicted in FIGS. 15-20 is that no headband is required to hold the housing assembly 12 over the pinna of the user's ear. Another advantage is the additional magnetic flux provided by the stack of ring magnets 58 within the ear mold 30B that combines with the flux provided by the magnets 22 in the housing assembly 12. Yet another advantage is that the ear mold 30B is a universal-fit design, which lowers its cost as compared to custom-molded ear molds.

### Method for Treatment of Sudden Sensorial Hearing Loss (SSNHL) Using Magnetic Fields

As described herein, magnetic therapy can enhance blood flow and oxygenation, reduce inflammation, and protect auditory nerve cells. For treatment of SSNHL, improving the blood supply to the cochlea aids in the recovery of hearing by ensuring that the hair cells and auditory nerve receive adequate nutrients and oxygen. Also, SSNHL is often associated with inflammation within the cochlea. Magnetic fields have been shown to have anti-inflammatory properties, which may help to reduce inflammation and edema in the inner ear, aiding in the restoration of hearing. Further, studies suggest that magnetic therapy has neuroprotective effects. Protecting the auditory nerve cells from further damage could be beneficial in preserving and potentially restoring hearing in patients with SSNHL.

In a preferred embodiment, treatment of SSNHL using magnetic therapy may be accomplished by the following steps:
(1) Placing a wearable appliance containing one or more magnets having a combined magnetic flux density in the range of 580 Gs to 1390 Gs (or more) on the head of a person to be treated such that the one or more magnets are in close proximity to one or both of the person's ears. The wearable appliance may be a configured as a headband, a headset, or in-ear inserts. If both of the person's ears are to be treated, the wearable appliance should include two sets of magnets disposed on opposite sides of the person's head and in close proximity to each of the person's inner ears.
(2) Orient the wearable appliance on the head or in the ears of the person to be treated, such that the north pole of the one or more magnets faces the person's inner ear, and the south pole of the one or more magnets faces away from the person's inner ear.
(3) Wear the appliance for as many hours as possible per day for a period of at least six weeks.

### Method for Treatment of Tinnitus Using Magnetic Fields

As described herein, magnetic therapy can enhance blood circulation, stimulate nerves in the ears, and reduce stress. Improving the blood supply to the inner ear may help reduce the severity of tinnitus symptoms. Another way magnetic therapy may help with tinnitus is by stimulating the nerves in the ear. The magnetic field generated by the magnets can interact with the nerves, possibly altering their activity. This stimulation might help to reduce the perception of tinnitus sounds or alleviate related discomfort. Further, tinnitus symptoms can often be exacerbated by stress and tension. Magnetic therapy sessions are typically relaxing, and many people report feeling calmer and more at ease afterward. By promoting relaxation and reducing stress levels, magnetic therapy may indirectly help to alleviate tinnitus symptoms.

In a preferred embodiment, treatment of tinnitus using magnetic therapy may be accomplished by the following steps:
(1) Placing a wearable appliance containing one or more magnets having a combined magnetic flux density in the range of 580 Gs to 1390 Gs (or more) on the head of a person to be treated such that the one or more magnets are in close proximity to one or both of the person's ears. The wearable appliance may be a configured as a headband, a headset, or in-ear inserts. If both of the person's ears are to be treated, the wearable appliance should include two sets of magnets disposed on opposite sides of the person's head and in close proximity to each of the person's inner ears.
(2) Orient the wearable appliance on the head or in the ears of the person to be treated, such that the north pole of the one or more magnets faces the person's inner ear, and the south pole of the one or more magnets faces away from the person's inner ear.
(3) Wear the appliance for as many hours as possible per day for a period of at least two weeks.

### Method for Treatment of Middle Ear Effusion/Otitis Media Using Magnetic Fields

Approximately 80% of all children will experience a case of otitis media during their lifetime, and between 80% and 90% of all children will have otitis media with fluid before school age. The common treatment for otitis media is high dose amoxycillin for ten days, in both children and adult patients. Amoxycillin has good efficacy in the treatment of otitis media due to its high concentration in the middle ear.

When an antibiotic pill or liquid enters the digestive tract, it is absorbed into the bloodstream, just as nutrients are from food. It circulates throughout the body and reaches its target area where bacteria are causing an infection. The blood supply to the middle ear primarily comes from a division of the external carotid artery, which is complemented by branches of the internal carotid artery. Magnetic treatment applied to the middle ear aligns blood molecules in the arteries, thereby bringing more oxygenation, along with any antibiotics, to an infected site.

In a preferred embodiment, treatment of middle ear effusion/otitis media using magnetic therapy may be accomplished by the following steps:
(1) Placing a wearable appliance containing one or more magnets having a combined magnetic flux density in the range of 580 Gs to 1390 Gs (or more) on the head of a person to be treated such that the one or more magnets are in close proximity to one or both of the person's ears. The wearable appliance may be a configured as a headband, a headset, or in-ear inserts. If both of the person's ears are to be treated, the wearable appliance should include two sets of magnets disposed on opposite sides of the person's head and in close proximity to each of the person's inner ears.
(2) Orient the wearable appliance on the head or in the ears of the person to be treated, such that the north pole of the one or more magnets faces the person's inner ear, and the south pole of the one or more magnets faces away from the person's inner ear.
(3) Wear the appliance for as many hours as possible per day for a period of at least two weeks.

### Method for Treatment of Temporomandibular Joint Disorders Using Magnetic Fields

Temporomandibular joint (TMJ) pain can be caused by many different factors such as osteoarthritis, jaw injury, stress causing grinding of the teeth, gum chewing, disc erosion, and genetics, to name a few. If the temporomandibular joint is injured or affected with a chronic condition, the joint and the surrounding tissue can become inflamed, which is the body's response to an irritant. One of the most common symptoms of temporomandibular joint disorder (TMD) is ear pain due to the fact that the middle ear and the joint are less than one inch apart. Although the inflammation starts in the TMJ, it can spread to surrounding tissues. In many cases, the pain and discomfort can be treated with self-managed care. Surgery may be considered as a last resort.

Pain in the ear is a common symptom for people with TMD. Because the TMJ is near the auditory canal, pain and inflammation in this joint can affect the ear. A ringing sound in the ear, also known as tinnitus, is often a part of TMD ear pain. TMD pain may be a dull, ongoing irritation or it may be experienced as a sharp, searing pain. This discomfort may be more apparent when the jaw moves to talk, chew, swallow, or yawn. In addition to experiencing ear and jaw pain, soreness may also be felt along the side of the head, neck, temple, cheek, face, lower jaw, and teeth.

Complementary treatment for TMD using magnetic therapy can ease symptoms and reduce recovery time. As described herein, magnetic therapy can enhance blood flow and oxygenation, provide nerve stimulation, reduce swelling, provide relaxation, and reduce stress. By placing magnets near the affected area, blood flow to the temporomandibular joint can be enhanced. This increased blood circulation helps reduce cellular swelling and relieves pain. Another way magnetic therapy may help with TMD symptoms is by calming the nervous system. The north magnetic field generated by magnets can interact with the nerves, altering their activity. This stimulation might help to reduce or alleviate related discomfort. Cellular swelling or edema causes inflammatory symptoms due to a lack of oxygen and acidity. A negative magnetic field reduces these conditions, thereby reversing inflammation and relieving the associated pain. Further, TMD symptoms can often be exacerbated by stress and tension. Magnetic therapy sessions are typically relaxing, and many people report feeling calmer and more at ease afterward. By promoting relaxation and reducing stress levels, magnetic therapy may indirectly help to alleviate TMD symptoms.

In a preferred embodiment, treatment of TMD using magnetic therapy may be accomplished by the following steps:
(1) Placing a wearable appliance containing one or more magnets having a combined magnetic flux density in the range of 580 Gs to 1390 Gs (or more) on the head of a person to be treated such that the one or more magnets are in close proximity to one or both of the person's ears. The wearable appliance may be a configured as a headband, a headset, or in-ear inserts. If the wearable appliance is in the form of a headset, it may be placed directly over the jaw to direct the magnetic field directly into the temporomandibular joint (TMJ).
(2) If both of the person's ears are to be treated, the wearable appliance should include two sets of magnets disposed on opposite sides of the person's head and in close proximity to each of the person's inner ears.
(3) Orient the wearable appliance on the head or in the ears of the person to be treated, such that the north pole of the one or more magnets faces the person's inner ear, and the south pole of the one or more magnets faces away from the person's inner ear.
(4) Wear the appliance for as many hours as possible per day for as long as possible, until the pain subsides.

### Method for Treatment of Vertigo Using Magnetic Fields

As described herein, magnetic therapy can improve blood flow, balance the nervous system, and reduce stress. Improving the blood supply to the inner ear may help reduce the frequency of vertigo symptoms. Another way magnetic therapy may help with vertigo is by stabilizing vestibular signals which may provide relief from dizziness. Further, vertigo symptoms can often be exacerbated by stress and tension. Magnetic therapy sessions may promote relaxation, thereby easing vertigo symptoms overall.

In a preferred embodiment, treatment of vertigo using magnetic therapy may be accomplished by the following steps:
1. Placing a wearable appliance containing one or more magnets having a combined magnetic flux density in the range of 580 Gs to 1390 Gs (or more) on the head of a person to be treated such that the one or more magnets are in close proximity to one or both of the person's ears. The wearable appliance may be a configured as a headband, a headset, or in-ear inserts. If both of the person's ears are to be treated, the wearable appliance should include two sets of magnets disposed on opposite sides of the person's head and in close proximity to each of the person's inner ears.
2. Orient the wearable appliance on the head or in the ears of the person to be treated, such that the north pole of the one or more magnets faces the person's inner ear, and the south pole of the one or more magnets faces away from the person's inner ear.
3. Wear the appliance for at least two hours per day for a period of at least two weeks.

The foregoing description of preferred embodiments for this invention have been presented for purposes of illustration and description. They are not intended to be exhaustive or to limit the invention to the precise form disclosed. Obvious modifications or variations are possible in light of the above teachings. The embodiments are chosen and described in an effort to provide the best illustrations of the principles of the invention and its practical application, and to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

1. An apparatus for directing a magnetic field to an inner ear of a user to improve blood flow and oxygen levels for treatment of one or all of tinnitus, sudden sensorineural hearing loss, vertigo, temporomandibular joint disorder, and middle ear effusion, the apparatus comprising:
a housing assembly configured to be positioned over a pinna of an ear of the user, the housing assembly comprising:
a base portion;
a cover portion configured to attach to the base portion, thereby forming a first cavity between the base portion and the cover portion; and
first magnetic material disposed within the first cavity, the first magnetic material having a north pole that is continuously directed toward a concha of the user's ear and a south pole that is continuously directed away from the concha of the user's ear when the housing assembly is disposed over the pinna of the ear of the user;
an ear mold attached to the housing assembly, the ear mold comprising:
an outer portion disposed adjacent the cover portion of the housing assembly, the outer portion configured to be disposed within the concha of the user's ear;
a second cavity disposed within the outer portion of the ear mold;
second magnetic material disposed within the second cavity of the outer portion, the second magnetic material having a north pole that is continuously directed toward the user's inner ear and a south pole that is continuously directed away from the user's inner ear when the outer portion is disposed within the concha of the user's ear; and
an inner portion attached to the outer portion, the inner portion sized and configured to be at least partially received within an ear canal of the user's ear,
wherein a force of magnetic attraction between the first magnetic material and the second magnetic material maintains the attachment of the housing assembly to the ear mold.

2. The apparatus of claim 1 further comprising:
the inner portion of the ear mold comprising a connector post; and
an ear tip connected to the connector post, the ear tip comprising a compliant outer structure disposed around a tubular core, wherein the compliant outer structure is configured to be received within the ear canal of the user.

3. The apparatus of claim 2 wherein the compliant outer structure of the ear tip comprises viscoelastic foam.

4. The apparatus of claim 2 wherein the connector post of the inner portion of the ear mold has external threads, and the tubular core of the ear tip has internal threads corresponding to the external threads of the connector post.

5. The apparatus of claim 1 wherein the outer portion of the ear mold is shaped and dimensioned to provide a universal fit that is applicable to more than one user.

6. The apparatus of claim 1 wherein:
the base portion includes first cradle protrusions extending from a central portion of an inner surface of the base portion, the first cradle protrusions disposed in a circular arrangement;
the first magnetic material comprises one or more circular magnetic disks disposed between and at least partially encircled by the first cradle protrusions; and
the cover portion includes second cradle protrusions extending from a central portion of an inner surface of the cover portion, the second cradle protrusions disposed in a circular arrangement and configured to have a snap-fit engagement with the first cradle protrusions.

7. The apparatus of claim 6 wherein the one or more circular magnetic disks comprise neodymium.

8. The apparatus of claim 6 wherein the one or more circular magnetic disks generate a magnetic flux density ranging from about 580 Gs to about 1390 Gs.

9. The apparatus of claim 1 wherein the first magnetic material comprises an electromagnet.

10. The apparatus of claim 1 wherein the second magnetic material comprises a stack of multiple annular ring magnets.

11. The apparatus of claim 10 wherein the stack of multiple annular ring magnets generate a magnetic flux density ranging from about 200 Gs to about 880 Gs.

12. The apparatus of claim 1 wherein the force of magnetic attraction between first magnetic material and the second magnetic material removably secures the ear mold to the housing assembly, the force of magnetic attraction being strong enough to maintain secure attachment of the housing assembly to the ear mold as the outer portion of the ear mold is placed into the user's ear concha and the inner portion of the ear mold is inserted into the user's ear canal, but the force of magnetic attraction being not strong enough to prevent the user from intentionally pulling the ear mold and the housing assembly apart when desired.

13. A method for using the apparatus of claim 1 to improve blood flow and oxygen levels within the ear of the user for treatment of one or more of tinnitus, sudden sensorineural hearing loss, vertigo, temporomandibular joint disorder, and middle ear effusion, the method comprising:
inserting the inner portion of the ear mold into the user's ear canal and inserting the outer portion of the ear mold into the user's ear concha such that the second magnetic material is in close proximity to the user's inner ear;
attaching the housing assembly to the ear mold such that the housing assembly is adjacent the pinna of the user's ear and the first magnetic material is in close proximity to the user's ear concha;
orienting the appliance such that the north poles of the first and second magnetic materials are directed toward the user's inner ear, and the south poles of the first and second magnetic materials are directed away from the user's inner ear; and
the user wearing the appliance for an extended period of time.

14. The method of claim 13 wherein the step of attaching the housing assembly to the ear mold is performed either before or after the steps of inserting the inner portion of the ear mold into the user's ear canal and inserting the outer portion of the ear mold into the user's ear concha.

15. A method for improving blood flow and oxygen levels within an ear of a user for treatment of one or more of tinnitus, sudden sensorineural hearing loss, vertigo, temporomandibular joint disorder, and middle ear effusion using an appliance containing magnetic material that generates a magnetic field, the method comprising:
placing the appliance on the user's head such that the magnetic material is in close proximity to one or both ears of the user;
orienting the appliance such that the north pole of the magnetic material is directed toward the user's ear, and the south pole of the magnetic material is directed away from the user's ear; and
the user wearing the appliance for an extended period of time.

16. The method of claim 15 wherein the extended period of time comprises at least two hours per day over a period of at least two weeks during which the magnetic material generates a magnetic flux density ranging from about 580 Gs to about 1390 Gs.

17. A method for improving blood flow and oxygen levels within an ear of a user for treatment of one or more of tinnitus, sudden sensorineural hearing loss, vertigo, temporomandibular joint disorder, and middle ear effusion using an in-ear appliance containing magnetic material that generates a magnetic field, the method comprising:
inserting the in-ear appliance into the user's ear such that the magnetic material is in close proximity to the inner ear of the user;
orienting the in-ear appliance such that the north pole of the magnetic material is directed toward the user's inner ear, and the south pole of the magnetic material is directed away from the user's inner ear; and
the user wearing the wearable appliance for an extended period of time.

18. The method of claim 17 wherein the inserting step comprises inserting the in-ear appliance into the user's ear canal.

19. The method of claim 17 wherein the inserting step comprises placing the in-ear appliance into the user's ear concha.

20. The method of claim 17 wherein the extended period of time comprises at least two hours per day over a period of at least two weeks during which the magnetic material generates a magnetic flux density ranging from about 580 Gs to about 1390 Gs or more.
